(19) United States
(12) Patent Application Publication
(10) Pub. No.: US 2015/0238296 A1
(43) Pub. Date: Aug. 27, 2015

(54) INJECTION OF AN ACTIVE INGREDIENT AND APPLICATION OF FLAVOR

(71) Applicant: Steven Sutter, Syosset, NY (US)

(72) Inventor: Steven Sutter, Syosset, NY (US)

(21) Appl. No.: 14/265,776

(22) Filed: Apr. 30, 2014

Related U.S. Application Data
(60) Provisional application No. 61/942,888, filed on Feb. 21, 2014.

(51) Int. Cl. A61C 19/08; A61M 5/19; A61C 19/06; A61K 31/245; A61K 31/167

(52) U.S. Cl. CPC A61C 19/08; A61K 31/245; A61K 31/167; A61C 19/063; A61M 5/19

(57) ABSTRACT

A device that supplies an active ingredient (e.g., for parenteral absorption) and enables a flavor to be orally dispensed (e.g., for enteral absorption) is provided. The device includes a flavor applicator and a carpule. The flavor applicator includes a chamber housing a flavored liquid, a plunger, and an oral dispenser. Depressing the plunger causes the flavored liquid to be orally dispensed through the oral dispenser. The carpule houses an active ingredient and the carpule is affixed to the chamber. The flavor applicator may be either permanently or removably attached to either the active ingredient carpule or an injection device that injects the active ingredient carpule.

### CROSS-REFERENCE TO RELATED APPLICATION

This application claims priority to U.S. Provisional Application Ser. No. 61/942,888 filed on Feb. 21, 2014.

### BACKGROUND OF THE INVENTION

1. Technical Field

The present disclosure relates generally to a device that enables an active ingredient to be injected (e.g., via parenteral absorption) and a flavor to be orally administered (e.g., via enteral absorption), and a method of using the device to inject the active ingredient and apply the flavor.

2. Discussion of Related Art

A dentist uses a needle to inject a local anesthetic into a part of the mouth to numb that part prior to performing a procedure (e.g., drilling/extracting a tooth). The local anesthetic may be considered an active ingredient and includes one or more numbing agents such as procaine, NOVOCAIN, lidocaine, etc. While a portion of the local anesthetic injected into the mouth is absorbed by the body (e.g., parenteral absorption), some remainder is unintentionally released into the mouth and makes contact with the tongue. However, the taste of the local anesthetic is typically unpleasant. Further, multiple injections may be required for a given procedure, which may exacerbate this unpleasant taste.

Thus, there is need for a device that enables an active ingredient to be injected to an oral site (e.g., location) that minimizes or eliminates this unpleasant taste.

### SUMMARY OF THE INVENTION

According to an exemplary embodiment of the invention, a device that supplies an active ingredient (e.g., for parenteral absorption) and enables a flavor to be orally dispensed (e.g., for enteral absorption) is provided. The device includes a flavor applicator having a chamber housing a flavored liquid, a plunger, and an oral dispenser, where depressing the plunger causes the flavored liquid to be orally dispensed through the oral dispenser. The device further includes a carpule housing an active ingredient, where the carpule is affixed to the chamber. The device is designed to allow the active ingredient and the flavor to be administered without coming into contact with one another during the administering process. After the active ingredient has been administered into tissue for parenteral absorption and the flavored liquid has been dispensed into the mouth for enteral absorption, some of the active ingredient may leak out of the tissue. However, since it will then come in contact with the flavored liquid within the mouth, the recipient of the administering process should not perceive

According to an exemplary embodiment of the invention, a device configured to dispense a flavor orally (e.g., enteral absorption) and mate with a carpule is provided. The device includes a flavor applicator having a chamber housing a flavored liquid, a plunger, and an oral dispenser, where depressing the plunger causes the flavored liquid to be orally dispensed through the oral dispenser. The device further comprises a connector permanently bonded to an outer side of the chamber and configured to mate with the carpule.

According to an exemplary embodiment of the invention, a device configured to inject an active ingredient (e.g., for parenteral absorption) and dispense a flavor orally (e.g., enteral absorption) is provided. The device includes an injection device having a chamber configured to receive a carpule having the active ingredient, and a flavor applicator having a chamber housing a flavored liquid, a plunger, and an oral dispenser, where depressing the plunger causes the flavored liquid to be orally dispensed through the oral dispenser, and an outer surface of the chamber is affixed to an outer surface of the injection device.

According to an exemplary embodiment of the invention, a method to inject an active ingredient (e.g., for parenteral absorption) and orally dispense a flavor (e.g., enteral absorption) is provided. The method includes affixing a flavor applicator housing a flavored liquid to a carpule housing an active ingredient; placing the carpule into a barrel of an injection device; using a plunger of the injection device to inject the active ingredient into oral tissue; and using a plunger of the flavor applicator to orally dispense the flavored liquid.

According to an exemplary embodiment of the invention, a method to inject an active ingredient (e.g., for parenteral absorption) and orally dispense a flavor (e.g., for enteral absorption) includes affixing a flavor applicator housing a flavored liquid to an outer surface of an injection device; placing a carpule housing an active ingredient into a barrel of the injection device; using a plunger of the injection device to inject the active ingredient into oral tissue; and using a plunger of the flavor applicator to orally dispense the flavored liquid.

The devices and methods described briefly above, and in more detail below, are designed to allow an active ingredient and a flavor to be administered without coming into contact with one another during the administering process. After the active ingredient has been administered into tissue for parenteral absorption and the flavored liquid has been dispensed into the mouth for enteral absorption, some of the active ingredient may leak out of the tissue. However, since it will then come in contact with the flavored liquid within the mouth, the recipient of the administering process should not perceive an unpleasant taste, or at the very least, the chances of the recipient perceiving an unpleasant taste should be substantially reduced.

### BRIEF DESCRIPTION OF THE DRAWINGS

Exemplary embodiments of the invention can be understood in more detail from the following descriptions taken in conjunction with the accompanying drawings in which:

FIG. 1 illustrates a device that supplies an injectable active ingredient (e.g., for parenteral absorption) and enables a flavor to be orally dispensed (e.g., for enteral absorption), according to an exemplary embodiment of the present invention;

FIG. 2 illustrates an injection device according to an exemplary embodiment of the invention that houses the device of FIG. 1;

FIG. 3 illustrates another side of the injection device of FIG. 2;

FIG. 4 illustrates a device that supplies an injectable active ingredient (e.g., for parenteral absorption) and enables a flavor to be orally dispensed (e.g., for enteral absorption), according to an exemplary embodiment of the present invention;

FIG. 5 illustrates a device that supplies an injectable active ingredient (e.g., for parenteral absorption) and enables a flavor to be orally dispensed, according to an exemplary embodiment of the present invention;

FIG. 6 illustrates a device that supplies an injectable active ingredient (e.g., for parenteral absorption) and enables a flavor to be orally dispensed (e.g., for enteral absorption), according to an exemplary embodiment of the present invention;

FIG. 7 illustrates a device that supplies an injectable active ingredient (e.g., for parenteral absorption) and enables a flavor to be orally dispensed (e.g., for enteral absorption), according to an exemplary embodiment of the present invention;

FIG. 8 illustrates a device that supplies an injectable active ingredient (e.g., for parenteral absorption) and enables a flavor to be orally dispensed (e.g., for enteral absorption), according to an exemplary embodiment of the present invention;

FIG. 9 illustrates a device that supplies an injectable active ingredient (e.g., for parenteral absorption) and enables a flavor to be orally dispensed (e.g., for enteral absorption), according to an exemplary embodiment of the present invention;

FIG. 10 illustrates a device that enables an active ingredient to be injected (e.g., for parenteral absorption) and a flavor to be applied (e.g., for enteral absorption), according to an exemplary embodiment of the present invention;

FIG. 11 illustrates a method to inject an active ingredient (e.g., for parenteral absorption) and orally dispense a flavor (e.g., for enteral absorption) according to an exemplary embodiment of the invention; and

FIG. 12 illustrates method to inject an active ingredient (e.g., for parenteral absorption) and orally dispense a flavor (e.g., for enteral absorption) according to an exemplary embodiment of the invention.

### DETAILED DESCRIPTION OF THE EXEMPLARY EMBODIMENTS

Exemplary embodiments of the present invention will be described below in more detail with reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

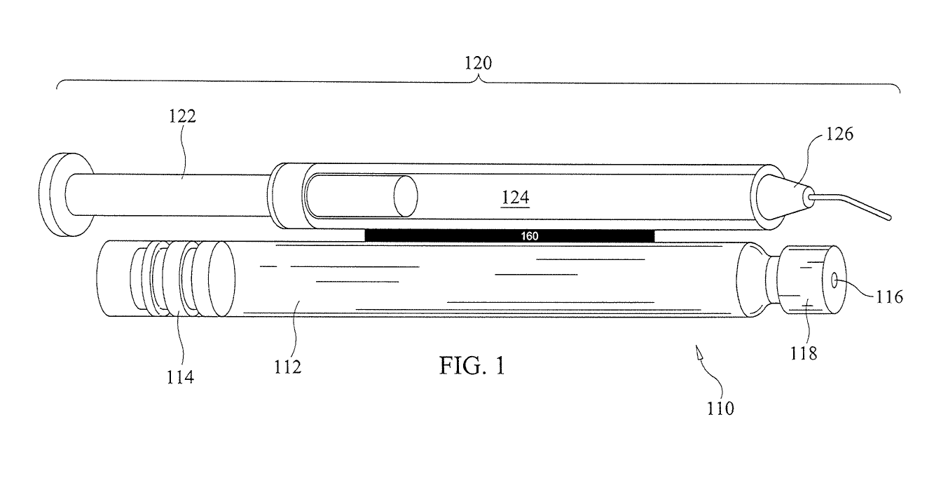

FIG. 1 illustrates a device that supplies an injectable active ingredient for parenteral absorption and enables a flavor to be orally dispensed for enteral absorption, according to an exemplary embodiment of the present invention;

Referring to FIG. 1, the device 100 includes a carpule 110 (e.g. a vial) affixed to a flavor applicator 120. The carpule 110 houses a liquid active ingredient 112 such as a local anesthetic, which is intended to be injected for parenteral absorption. A movable stopper 114 (e.g., made of rubber) in contact with the active ingredient 112 is located at one end (e.g., an open end) of the carpule 110. The other end of the carpule 110 includes a cap 118 with a hole 116 that may be covered with a piercable membrane.

The flavor applicator 120 includes a depressible plunger 122, a flavor reservoir 124 (e.g., a chamber) housing a flavored liquid, and a flavor dispenser 126. The flavored liquid is intended for enteral absorption. In an exemplary embodiment, the flavored liquid is an inactive ingredient as no active ingredients are housed within the flavor reservoir 124.

The end of the flavor dispenser 126 may be narrower than the rest of the flavor dispenser 126. The end of the flavor dispenser 126 has an opening, is not sharp, and accordingly is not meant to pierce tissue, but to enable the flavored liquid to be orally dispensed in a concentrated stream out of its opening. Depressing the plunger 122 causes the flavored liquid to be evacuated via the flavor dispenser 126 so that a flavor can be dispensed orally (e.g., to a location within the mouth). The flavor reservoir 124 of the flavor applicator 120 may include a removable stopper (not shown) that enables it to be refilled.

As shown in FIG. 1, an adhesive 160 (e.g., glue or other bonding agent) is used to permanently bond the flavor reservoir 124 of the flavor applicator 120 to the carpule 110.

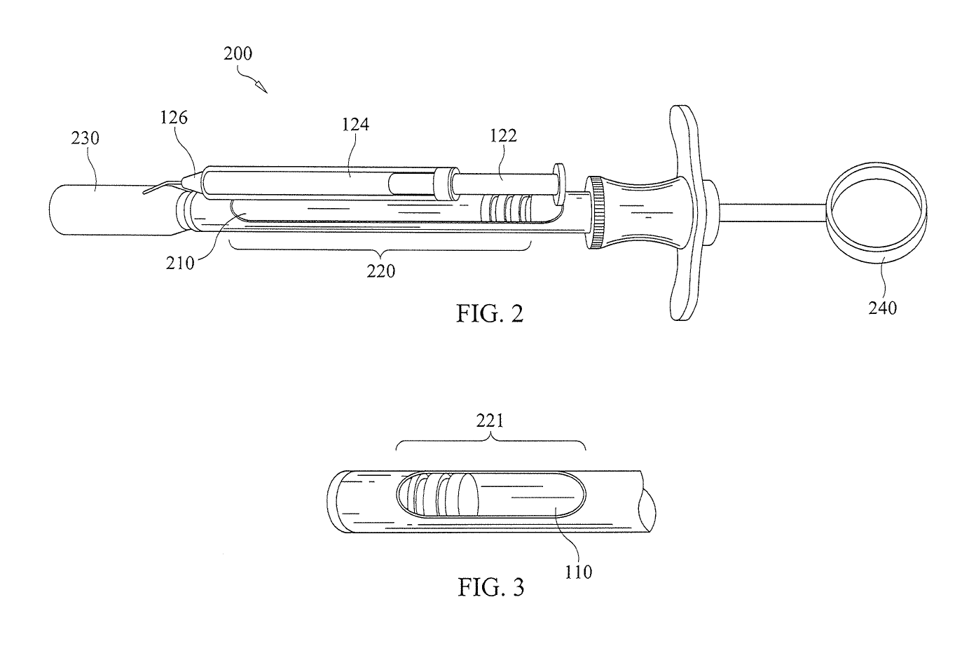

FIG. 2 illustrates an injection device 200 (e.g., a syringe with a needle) that houses the device of FIG. 1. In an exemplary embodiment of the invention, the injection device 200 is a submucosal needle. The carpule 110 portion of the device of FIG. 1 is placed in an opening 210 of a barrel 220 of the injection device 200 so that the flavor applicator 120 extends outside the opening 210.

The injection device 200 includes a depressible piston rod (not shown) that is applied to the stopper 114 when the O-Ring 240 is depressed to evacuate the active ingredient 112 through the opening 116 through a needle (not shown) within the needle protector 230. The plunger 122 of the flavor applicator 120 can be depressed independent of the O-Ring 240.

For example, at the same time or after the active ingredient 112 has been injected into a part of the mouth, while at least a portion of the injection device 200 is still located within or near the injection site (e.g., the gum line), the plunger 122 can be pressed to orally dispense the flavored liquid to an interior of the mouth. While the taste of the injected active ingredient may be unpleasant, since the flavored liquid is applied at the same time the injection is applied, soon after the injection is applied, or soon before the injection is applied, the patient should not experience this unpleasant taste, or at the very least the chances of the patient experiencing this unpleasant taste are reduced.

As discussed above, the flavored liquid housed in the flavor reservoir 124 in the above embodiment is an inactive ingredient. In an alternate embodiment, the flavored liquid not only comprises an inactive ingredient such as a flavoring, but an active ingredient such as an oral antibiotic or a topical local anesthetic.

FIG. 3 shows the opposite side of a portion of the injection device 200 that houses the carpule 110. The opposite side includes an opening 221 that enables the carpule 110 that is affixed to the flavor applicator 120 to be popped out of the barrel 220.

While FIG. 1 shows the flavor applicator 120 permanently bonded to the vial 110 using an adhesive 160, in alternate embodiments of the invention, other mechanisms are used to secure the flavor applicator 120 to the carpule 110.

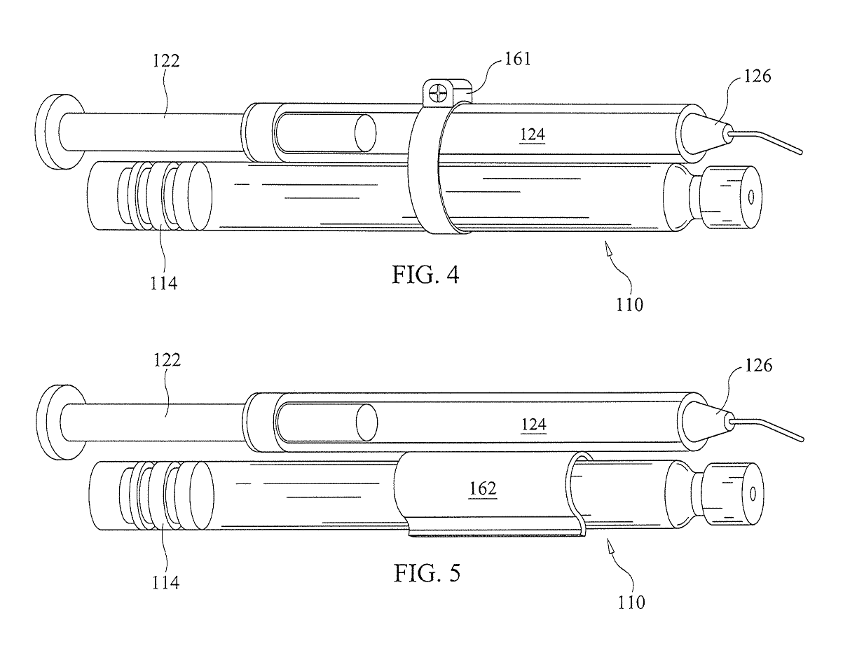

FIG. 4 illustrates a device that supplies an injectable active ingredient and enables a flavor to be orally dispensed, according to an exemplary embodiment of the present invention.

As shown in FIG. 4, a clamp 161 is tightened around both the flavor reservoir 124 and the carpule 100. In an exemplary embodiment, an inner surface of the clamp 161 is permanently bonded to a side of the flavor reservoir 124, and the carpule 110 can be removed when the clamp 161 is loosened. Thus, the carpule 110 can be manufactured separately from a device comprising the flavor applicator 120 and the clamp 161. As an example, the clamp 161 could be made of metal, plastic, rubber, etc.

The device shown in FIG. 4 is configured to be placed into the opening 210 of the barrel 220 of the injection device 200 of FIG. 2. For example, the carpule 110 which is surrounded by a portion of the clamp 161 is configured to be placed inside the opening 210 of the barrel so that the flavor applicator 120 extends outside the opening 210.

In a variation of FIG. 4, the clamp 161 is replaced with a continuous rubber band with a stretched circumference that is just large enough to fit around both the reservoir 124 and the carpule 110. For example, one end of the rubber band is fused to the outer side of the reservoir 124, the rubber band is stretched around the carpule 110, and carpule 110 can be removed.

In another variation of FIG. 4, the clamp 161 is replaced with a twistable length of metal with open and opposite ends, where a part of the metal between the ends is fused to the outer side of the reservoir 124 and the ends are twisted around the carpule 110.

FIG. 5 illustrates a device that supplies an injectable active ingredient and enables a flavor to be orally dispensed, according to an exemplary embodiment of the present invention.

As shown in FIG. 5, the device includes a carpule 110 affixed to the flavor applicator 120 via a grip 162. The grip 162 may be U-shaped, an open cylinder, etc. For example, a cylinder or a cylindrical like object could be cut along its length to create two grips.

The grip 162 is mounted to a side of the flavor reservoir 124 and the carpule 110 fits within the curved inner surface of the grip 162. For example, if the grip 162 is U-shaped, a bottom outer side of the U-shape is affixed to an outer side of the flavor reservoir 124, and the open inner side of the U-shape is configured to surround the carpule 110.

The diameter of the grip 162 un-expanded is less than the diameter of the carpule 110 and pressing the carpule 110 into the grip 162 expands the diameter of the grip 162 enough to allow the carpule 110 to fit snugly and securely within the grip 162. When the carpule 110 needs to be replaced, it can be popped out of the grip 162. The grip 162 may be made of a compressible/flexible plastic as an example.

The device shown in FIG. 5 is configured to be placed into the opening 210 of the barrel 220 of the injection device 200 of FIG. 2. For example, the carpule 110 which is surrounded by the grip 162 is configured to be placed inside the opening 210 of the barrel so that the flavor applicator 120 extends outside the opening 210.

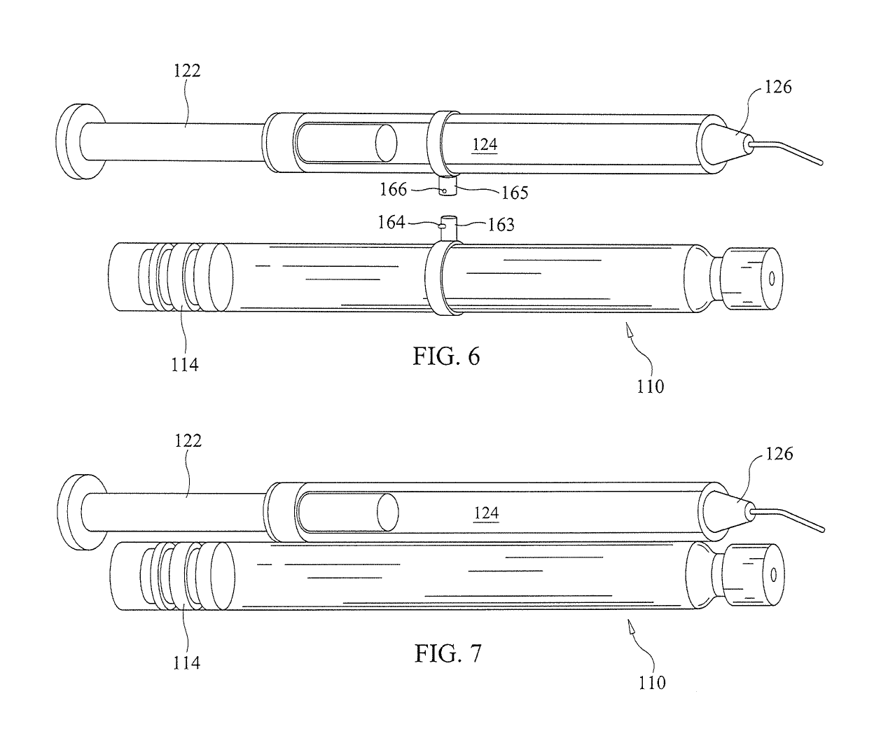

FIG. 6 illustrates a device that supplies an injectable active ingredient and enables a flavor to be orally dispensed, according to an exemplary embodiment of the present invention.

As shown in FIG. 6, a first band with a first extension 163 surrounds the carpule 110, a second band with a second extension 165 surrounds the flavor reservoir 124 of the flavor applicator 120 and the extensions snap into one another. The extensions 163 and 165 may be cylindrical in shape and hollow. For example, the first extension 163 includes a depressible button 164 that fits into a through-hole 166 of the second extension 165. Alternately the through-hole 166 is disposed on the first extension 163 and the button 164 is disposed on the second extension 165. Thus, when either a new carpule 110 or a new flavor applicator 120 is needed, the respective element can be easily disconnected from the other component for replacement. The second band may be permanently fused around the flavor reservoir 124, and then the carpule 110 can be replaced by pushing it through the first band. Each band may also include a pressure clasp that allows each band to be clamped onto a given carpule or reservoir, or un-clamped from the same. In an alternate embodiment, the first and second extensions 163 and 165 are permanently fused together into a single extension.

The device shown in FIG. 6 is configured to be placed into the opening 210 of the barrel 220 of the injection device 200 of FIG. 2. For example, the carpule 110 which is surrounded by the first band and extension 163 is configured to be placed inside the opening 210 of the barrel so that the flavor applicator 120 extends outside the opening 210.

FIG. 7 illustrates a device that supplies an injectable active ingredient and enables a flavor to be orally dispensed, according to an exemplary embodiment of the present invention.

As shown in FIG. 7, the carpule 110 and the reservoir 124 are one integral component or one continuous piece. The one continuous piece includes two chambers that are sealed (separated) from each other, where the upper chamber houses the active ingredient and the lower chamber houses the flavored liquid. Thus, the fluid in the carpule 110 does not mix with the fluid in the reservoir 124.

The device shown in FIG. 7 is configured to be placed into the opening 210 of the barrel 220 of the injection device 200 of FIG. 2. For example, the carpule 110 is configured to be placed inside the opening 210 of the barrel so that the flavor applicator 120 extends outside the opening 210.

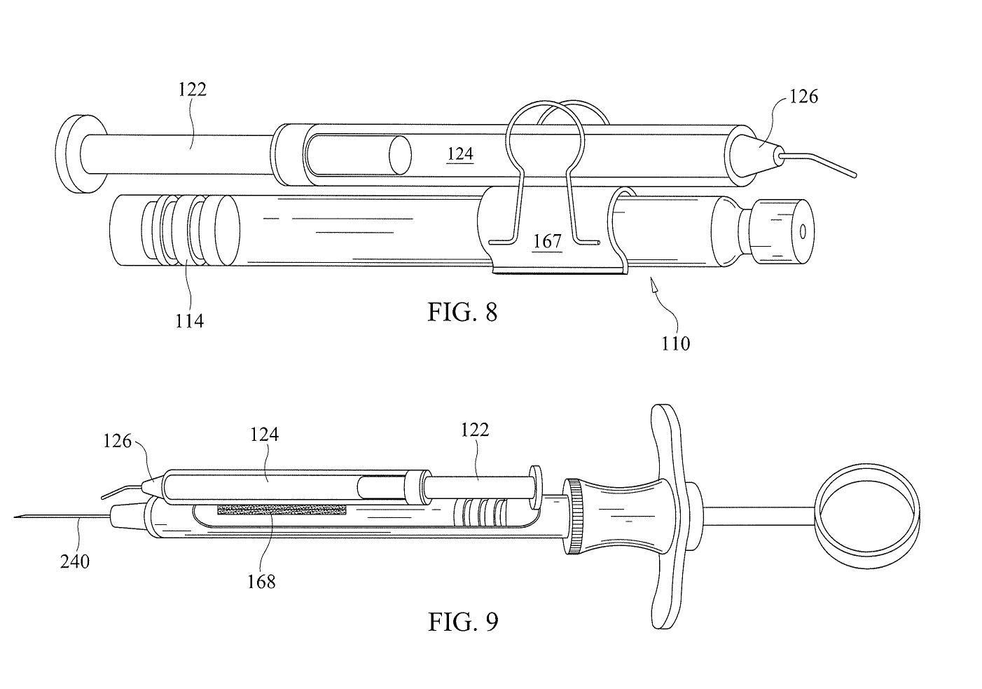

FIG. 8 illustrates a device that supplies an injectable active ingredient and enables a flavor to be orally dispensed, according to an exemplary embodiment of the present invention.

As shown in FIG. 8, a clip 167 is permanently affixed to the reservoir 124 of the flavor applicator 120 and the clip 167 clips around the carpule 110. For example, a side of the clip 167 opposing an opening of the clip 167 that mates with carpule 110 is bonded to a side of the reservoir 124. The carpule 110 can then be released by applying pressure to a pair of extensions of the clip 167 illustrated in FIG. 8 as looped wire portions.

The device shown in FIG. 8 is configured to be placed into the opening 210 of the barrel 220 of the injection device 200 of FIG. 2. For example, the carpule 110 which is surrounded by a portion of the clip 167 is configured to be placed inside the opening 210 of the barrel so that the flavor applicator 120 extends outside the opening 210.

FIG. 9 illustrates a device that supplies an injectable active ingredient and enables a flavor to be orally dispensed, according to an exemplary embodiment of the present invention.

As shown in FIG. 9, the flavor applicator 120 is affixed to the carpule 110 using VELCRO strips 168. In an exemplary embodiment of the invention, the label 168 can also represent magnets or magnetic strips but shall be referred to as VELCRO strips below. The strips 168 include a first strip comprising VELCRO hooks or loops, where the first strip is bonded to a side of the reservoir 124 of the flavor applicator 120, and a second strip comprising mating VELCRO hooks or loops opposite to the first strip is bonded to a side of the carpule 110. In an exemplary embodiment, each strip has a square or rectangular shape. The flavor applicator 120 is then secured to the carpule 110 by pressing the VELCRO strip of the reservoir 124 against the VELCRO strip of the carpule 110. When the strips 168 are magnets or magnetic strips, they are of different polarities so that they stick together.

The device shown in FIG. 9 is configured to be placed into the opening 210 of the barrel 220 of the injection device 200 of FIG. 2. For example, a side of the carpule 110 opposing the side of the carpule 110 that is affixed to the VELCRO strip is configured to be placed inside the opening 210 of the barrel 220 so that the flavor applicator 120 extends outside the opening 210.

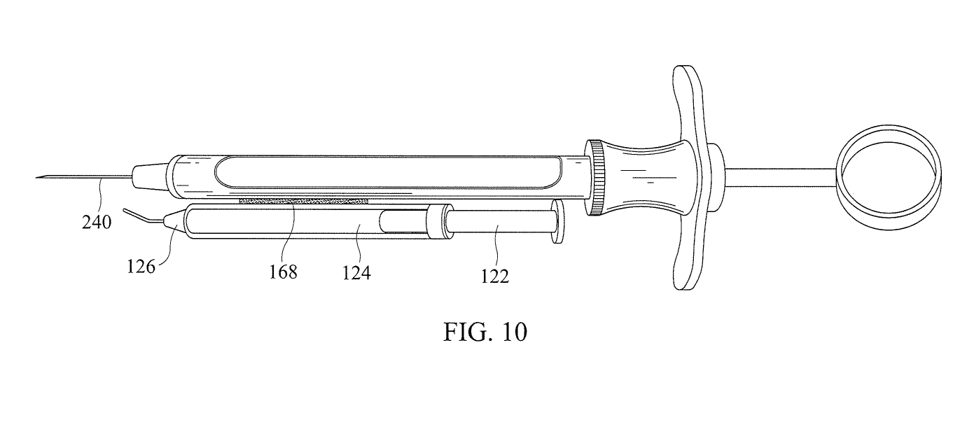

FIG. 10 illustrates a device that enables an active ingredient to be injected and a flavor to be applied, according to an exemplary embodiment of the present invention.

As shown in FIG. 10, the flavor applicator 120 is affixed to the outer metal side of the injection device 200 using VELCRO strips 168. The strips 168 include a first strip comprising VELCRO hooks or loops, where the first strip is bonded to a side of the reservoir 124 of the flavor applicator 120, and a second strip comprising mating VELCRO hooks or loops opposite to the first strip is bonded to the outer metal side of the injection device 200. The flavor applicator 120 is then secured to the injection device 200 by pressing the VELCRO strip of the reservoir 124 against the VELCRO strip of the injection device 200.

In an exemplary embodiment, the first VELCRO strip is bonded to a side of the injection device 200 that opposes the opening of the chamber of the injection device 200 that is configured to house a carpule. At the very least the first VELCRO strip is bonded to a location of the injection device 200 such that when the second VELCRO strip of the flavor applicator 120 is mated with the first VELCRO strip, the flavor applicator 120 does not overlap the opening of the barrel housing the carpule 110. Thus, the flavor applicator 120 does not impede insertion or removal of the carpule 110.

In an exemplary embodiment of the invention, the strips 168 are not VELCRO, but magnets of opposite polarities.

Further, the flavor applicator 120 may be affixed to the outer metal side of the injection device 200 with any of the previously described or illustrated mechanisms for removably connecting or bonding the flavor applicator 120 to the carpule 110. Moreover, the flavor applicator 120 may be affixed to the outer metal side of the injection device 200 or to the carpule 100 using other techniques not described above.

In an exemplary embodiment, the strips 168 illustrated in FIG. 10 are omitted, and the clamp 161 of FIG. 4 wraps around both the flavor applicator 120 and the body of injection device 200. In this embodiment, the clamp 161 surrounds a portion of the injection device 200 without overlapping the opening of the injection device 200 that receives the carpule 110. An example of this portion is the metal portion between the needle 240 and the opening or the metal portion between the finger grip and the opening.

In an exemplary embodiment, the strips 168 illustrated in FIG. 10 are omitted, and the closed side of the grip 162 of FIG. 5 is affixed (e.g., permanently bonded) to the above described metal portion of the injection device (e.g., the portion that does not overlap the opening receiving the carpule 100) and the grip 162 grips the flavor applicator 120. For example, in FIG. 5, the closed side of the grip 162 is affixed to the flavor applicator 120 and the carpule 110 is pressed into the open side of the grip 162, whereas in this embodiment, the closed side of the grip 162 is affixed to the injection device 200 and the flavor applicator 120 is pressed into the open side of the grip 162.

In an exemplary embodiment, the strips 168 illustrated in FIG. 10 are omitted, and the rings and extensions (e.g., 163-165) of FIG. 6 are used to secure the flavor applicator 120 to the injection device 200. For example, the ring with extension 163 of FIG. 6 is fitted around the metal portion of injection device 200 (e.g., the portion that does not overlap the opening that receives the carpule 110) shown in FIG. 10, the ring with extension 165 of FIG. 6 is fitted around the flavor applicator 120 shown in FIG. 10, and then the extensions 163 and 165 are snapped together.

In an exemplary embodiment, the strips 168 illustrated in FIG. 10 are omitted, and the closed side of the clip 167 of FIG. 5 is affixed (e.g., permanently bonded) to the above described metal portion of the injection device (e.g., the portion that does not overlap the opening receiving the carpule 100) and the clip 167 grips the flavor applicator 120. For example, in FIG. 8, the closed side of the clip 167 is affixed to the flavor applicator 120 and the carpule 110 is pressed into the open side of the clip 167, whereas in this embodiment, the closed side of the clip 167 is affixed to the injection device 200 and the flavor applicator 120 is pressed into the open side of the clip 167.

In an exemplary embodiment of FIG. 10, the flavor applicator 120 is permanently bonded to the outer metal side of the injection device 200 using adhesive 160.

When the flavor applicator 120 is permanently bonded to the injection 200 device it may be manufactured along with the injection device 200 as one integrated unit. When the flavor applicator 120 is connected to the injection device 200 with the above described mechanisms that allow them to be removed from one another, they can be manufactured separately.

While the length of the plunger 122 is shown as not extending up to the finger grip of the injection device 200 in the figures, its length may vary in alternate embodiments. For example, the plunger 122 may extend beyond the O-ring 240, may be level with the O-ring 240, may extend between the finger grip and the O-ring 240, be level with the finger grip, etc. Further, various different types of injection devices 200 may be used. For example, the hollow O-ring 240 may be replaced with a solid circular portion, a solid non-circular portion, or may be omitted entirely, so long as there is some way to drive the internal piston of the injection device 200.

The shaft between the O-Ring 240 and the finger grip drives an internal piston rod into the stopper 114 of the carpule 110. In an exemplary embodiment of the invention, the plunger 122 is attached to the shaft or the O-Ring 240 so that at the same time the injection device 200 injects the active ingredient, the flavor applicator 120 orally dispenses the flavor. For example, a first end of a rigid length of rigid material (e.g., rigid metal, rigid plastic) is affixed to the plunger 122 and a second other end of the rigid material is affixed to the shaft so that the flavor is applied at the same time the injection is applied.

In an exemplary embodiment of the invention, at least one of the above-described devices is used in a dental setting to numb an area of the mouth and orally dispense a flavor, where the carpule 110 is pre-filled with a local anesthetic such as NOVOCAINE, lidocaine, etc., to act as the active numbing agent.

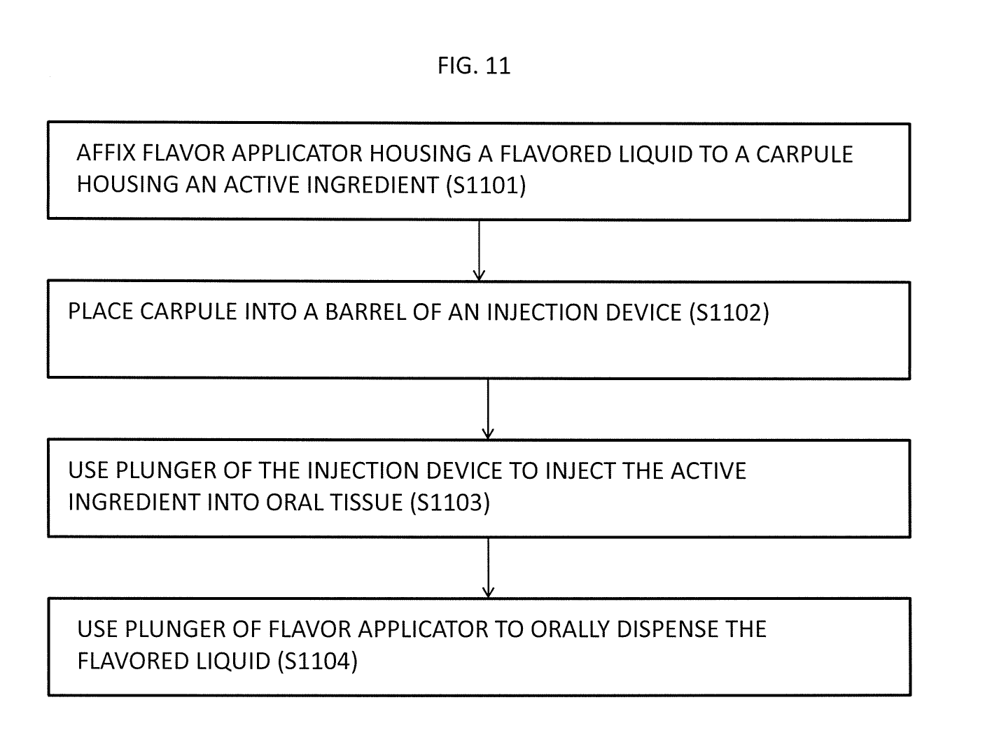

As shown in FIG. 11, a method to inject an active ingredient and orally dispense a flavor according to an exemplary embodiment of the invention includes: affixing a flavor applicator housing a flavored liquid to a carpule housing an active ingredient (S1101), placing the carpule into a barrel of an injection device (S1102), using a plunger of the injection device to inject the active ingredient into oral tissue (S1103), and using a plunger of the flavor applicator to orally dispense the flavored liquid (S1104). Steps 1103 and 1104 may happen at the same time, at substantially the same time, or one of the steps 1103 and 1104 may precede the other. For example, the flavor could be orally dispensed before the active ingredient is injected, or vice versa.

When the flavor applicator 120 has been permanently affixed to the carpule 110 by the manufacturer, the step of affixing the flavor applicator 120 to the carpule 110 is omitted by the medical professional performing the above method. If the flavor applicator 120 is removably attached to the carpule 110, the step of affixing the flavor applicator 120 depends on which of the above embodiments is employed. For example, when the device of FIG. 9 is employed, the step of affixing the flavor applicator 120 includes pressing a VELCRO covered side of the flavor applicator 120 against a VELCRO covered side of the carpule 110.

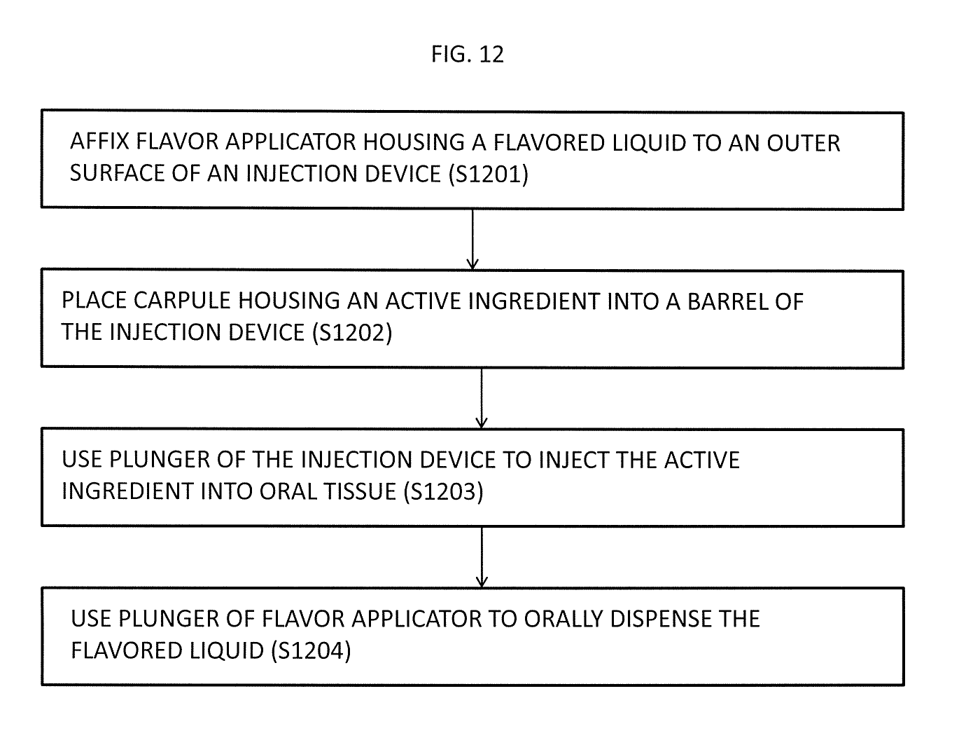

FIG. 12 illustrates a method for using the device of FIG. 10 to inject an active ingredient and orally dispense a flavor according to an exemplary embodiment of the invention. Referring to FIG. 12, the method includes: affixing a flavor applicator 120 housing a flavored liquid to an outer surface of an injection device (S1201), placing a carpule housing an active ingredient into a barrel of the injection device (S1202), using a plunger of the injection device to inject the active ingredient into oral tissue (S1203), and using a plunger of the flavor applicator to orally dispense the flavored liquid (S1204). Steps 1203 and 1204 may happen at the same time, at substantially the same time, or one of the steps 1203 and 1204 may precede the other. For example, the flavor could be orally dispensed before the active ingredient is injected, or vice versa.

When the flavor applicator 120 has been permanently affixed to the outer metal side of the injection device 200 by the manufacturer, the step of affixing the flavor applicator 120 to the outer side of the injection device is omitted by the medical professional performing the above method. If the flavor applicator 120 is removably attached to the outer side of the injection device 200 using VELCRO, the step of affixing the flavor applicator 120 includes pressing a VELCRO covered side of the flavor applicator 120 against a VELCRO covered side of the injection device 200.

While the injection device 200 has been described above as being made of metal, other materials may be used.

While FIGS. 9 and 10 illustrates the injection device 200 with the needle protector 230 removed so that the needle 240 is visible and FIG. 2 illustrates the needle protector 230 covering the needle 240, the injection device 200 in any of the illustrated embodiments may omit or include the needle protector 230.

Although the illustrative embodiments have been described herein with reference to the accompanying drawings, it is to be understood that the present invention is not limited to those precise embodiments, and that various other changes and modifications may be affected therein by one of ordinary skill in the related art without departing from the scope or spirit of the invention. All such changes and modifications are intended to be included within the scope of the disclosure.

1. A device that supplies an active ingredient for parenteral absorption and enables a flavor to be orally dispensed for enteral absorption, the device comprising:
a flavor applicator comprising a chamber housing a flavored liquid, a plunger, and an oral dispenser, wherein depressing the plunger causes the flavored liquid to be orally dispensed through the oral dispenser; and
a carpule housing an active ingredient, wherein the carpule is affixed to the chamber.

2. The device of claim 1, wherein the active ingredient is a local anesthetic.

3. The device of claim 1, wherein an adhesive permanently bonds the carpule to the chamber.

4. The device of claim 1, further comprising a clamp that surrounds the chamber and the carpule.

5. The device of claim 1, further comprising a flexible u-shaped grip, wherein a bottom outer side of the u-shape opposing an opening of the u-shape is permanently bonded to an outer side of the chamber, the carpule is surrounded by the grip within the opening, and the carpule is removable.

6. The device of claim 1, further comprising:
a first band that surrounds the carpule and a first extension that extends from the first band;
a second band that surrounds the chamber and second extension that extends from the second band,
wherein the first extension removably mates with the second extension.

7. The device of claim 6, wherein one of the extensions has a depressible button that fits into a through-hole in the other extension.

8. The device of claim 1, wherein the chamber and the carpule is one single continuous integrated piece, where the flavored liquid is isolated from the active ingredient.

9. The device of claim 1, further comprising a flexible u-shaped clip, wherein a bottom outer side of the u-shape opposing an opening of the u-shape is permanently bonded to an outer side of the chamber, the carpule is surrounded by the clip, and the clip includes a pair of extensions, where pressure applied to the extensions to enable the opening to be widened to remove the carpule.

10. The device of claim 1, further comprising a first VELCRO strip permanently affixed to an outer side of the chamber that mates with a second VELCRO strip permanently affixed to an outer side of the carpule.

11. (canceled)

12. A device configured to dispense a flavor orally and mate with a carpule, the device comprising:
a flavor applicator comprising a chamber housing a flavored liquid, a plunger, and an oral dispenser, wherein depressing the plunger causes the flavored liquid to be orally dispensed through the oral dispenser; and
a connector permanently bonded to an outer side of the chamber and configured to mate with the carpule.

13. The device of claim 12, wherein the connector is a clamp whose inner surface is permanently bonded to an outer surface of the chamber.

14. The device of claim 12, wherein the connector is a flexible u-shaped grip, wherein a bottom outer side of the u-shape opposing an opening of the u-shape is permanently bonded to an outer side of the chamber.

15. The device of claim 12, wherein the connector comprises a first ring that surrounds the chamber and a second ring.

16. The device of claim 15, wherein the first ring is affixed to the second ring via an extension.

17. The device of claim 12, wherein the connector is a flexible u-shaped clip, wherein a bottom outer side of the u-shape opposing an opening of the u-shape is permanently bonded to an outer side of the chamber and the clip includes a pair of extensions, where pressure applied to the extensions enables the opening to be widened to remove the carpule.

18. The device of claim 12, wherein the connector is a VELCRO strip permanently affixed to an outer side of the chamber.

19. A device configured to inject an active ingredient for parenteral absorption and dispense a flavor orally for enteral absorption, the device comprising:
an injection device comprising a chamber configured to receive a carpule comprising the active ingredient; and
a flavor applicator comprising a chamber housing a flavored liquid, a plunger, and an oral dispenser, wherein depressing the plunger causes the flavored liquid to be orally dispensed through the oral dispenser,
wherein an outer surface of the chamber is affixed to an outer surface of the injection device.

20. The device of claim 19, wherein the active ingredient is a local anesthetic.

21. The device of claim 19, wherein the chamber is affixed to the outer surface such that the flavor applicator does not overlap an opening of the chamber.

22. The device of claim 19, wherein the outer surface of the chamber is permanently affixed to the outer surface of the injection device.

23. The device of claim 19, further comprising:
a first strip of VELCRO permanently bonded to the outer surface of the chamber; and
a second strip of VELCRO permanently bonded to the outer surface of the injection device, wherein the first strip mates with the second strip.

24. (canceled)

25. A method to inject an active ingredient for parenteral absorption and orally dispense a flavor for enteral absorption, the method comprising:
affixing a flavor applicator housing a flavored liquid to a carpule housing an active ingredient;
placing the carpule into a barrel of an injection device;
using a plunger of the injection device to inject the active ingredient into oral tissue; and
using a plunger of the flavor applicator to orally dispense the flavored liquid.

26. The method of claim 25, wherein the active ingredient is a local anesthetic.

27. A method to inject an active ingredient for parenteral absorption and orally dispense a flavor for enteral absorption, the method comprising:
affixing a flavor applicator housing a flavored liquid to an outer surface of an injection device;
placing a carpule housing an active ingredient into a barrel of the injection device;
using a plunger of the injection device to inject the active ingredient into oral tissue; and
using a plunger of the flavor applicator to orally dispense the flavored liquid.

28. The method of claim 27, wherein the active ingredient is a local anesthetic.
